(19) United States
(12) Patent Application Publication
(10) Pub. No.: US 2005/0180919 A1
(43) Pub. Date: Aug. 18, 2005

(54) Stent with radiopaque and encapsulant coatings

(76) Inventor: Eugene Tedeschi, Sant Rosa, CA (US)

Correspondence Address: MEDTRONIC VASCULAR, INC.;IP LEGAL DEPARTMENT, 3576 UNOCAL PLACE, SANTA ROSA, CA, 95403, US

(21) Appl. No.: 10/778,974

(22) Filed: Feb. 12, 2004

(51) Int. Cl. A61K 49/04; A61M 31/00

(52) U.S. Cl. 424/9.4; 604/500

(57) ABSTRACT

The present invention provides a system for treating a vascular condition, including a catheter, a stent having a stent framework coupled to the catheter, a radiopaque oxide coating substantially covering at least an outer perimeter portion of the stent framework, and an encapsulant coating disposed on the radiopaque oxide coating. A drug-coated stent with a radiopaque oxide coating and a method of manufacturing are also disclosed.

### FIELD OF THE INVENTION

This invention relates generally to biomedical stents. More specifically, the invention relates to a radiopaque oxide coating on a stent framework for a drug-polymer coated stent.

### BACKGROUND OF THE INVENTION

Implantable biomedical stents are typically formed from metallic or polymeric materials, and are deployed in the body to reinforce blood vessels and other vessels within the body as part of surgical procedures that require enlargement and stabilization of the lumens. With generally open tubular structures, stents typically have apertured or lattice-like walls, and can be either balloon expandable or self-expanding. A stent is usually deployed by mounting the stent on a balloon portion of a balloon catheter, positioning the stent in a body lumen, and expanding the stent by inflating the balloon. The balloon is then deflated and removed, leaving the stent in place.

A desirable endovascular stent provides an ease of delivery and necessary structural characteristics for vascular support, as well as long-term biocompatibility, antithrombogenicity, and antiproliferative capabilities. Stents are being coated with protective materials such as polymers to prevent corrosion, and with bioactive agents and drug polymers to help reduce tissue inflammation, thrombosis and restenosis at the site being supported by the stent.

Stents need to be radiopaque as well as biocompatible and corrosion-resistant. The proper deployment of a stent requires that a medical practitioner be able to follow the movement of a stent through the body vasculature to precisely position the device at the affected site. Determining the position of stents with fluoroscope or x-ray monitoring equipment can be difficult in that the devices are not always easily seen. For improved visibility, some stents have been designed to include radiopaque markers of palladium, platinum, tungsten, platinum-iridium, rhodium, gold, or other heavy metals that block the transmission of x-rays. As a result, they appear as contrasting images against the background of the fluoroscope or x-ray imaging equipment.

The opacity, degree of contrast, and sharpness of the stent image varies with the material and type of process used to create the stent as well as the additional radiopaque markers. The radiopacity of the stent in particular may be limited with some metals such as stainless steel and nitinol, particularly when struts of the stents are made thinner or spaced farther apart. Additional radiopaque markers may be included as bands around one or more struts or as rivets attached to the strut framework. Radiopaque stent markers are described, for example, in “Radiopaque Stent Markers” U.S. Pat. No. 6,402,777 by Globerman, et al. issued Jun. 11, 2002. Yet these markers only enhance the visibility of limited regions such as the ends of the stent, provide limited information about stent diameter, and can present electrochemical potentials that lead to undesirable corrosion after deployment.

A stent with a radiopaque core to enhance the resolution of the stent under fluoroscopy is described in “Vascular Stent having Increased Radiopacity and Method for Making Same” by Dang, U.S. Pat. No. 6,471,721 issued Oct. 29, 2002, though radiopaque materials in the core do not always offer the desired mechanical properties for self-expanding or balloon-deployed stents.

Stents may have coatings to reduce thrombosis and other effects when the base metal is exposed to the host. A stent comprising a single homogeneous tubing of niobium with a surface coating of iridium oxide or titanium nitrate to inhibit closure of a vessel at a site of stent implant is described in “Vascular and Endoluminal Stents” by Alt, U.S. Pat. No. 6,478,815 issued Nov. 12, 2002. Radiopaque coatings, however, may be more reactive or fragile—whether chemically, mechanically or biologically—to the relevant environment than desired as compared to the otherwise untreated surface of the underlying stent.

For example unwanted chemical reactions to the radiopaque coating may arise from the chemicals used to coat the stent with a therapeutic agent, including any polymers, solvents, preservatives or additives used. The use of preservatives such as BHT in a stent coating, for example, are disclosed in Carlyle et al App. Ser. No. 10/133,181 entitled “Endovascular Stent With A Preservative Coating” filed Apr. 26, 2002, incorporated herein by reference. Further chemical reactions may arise during sterilization, including due to the use of chemical, radiation, e-beam or other methods of sterilizing. Unwanted mechanical alterations to the radiopaque coating may arise during the handling of the coated stent, including during any of the steps of mounting the stent to the delivery catheter, packaging the system (stent and catheter) as well as introducing the stent to the desired anatomical location. Unwanted biologic interactions may arise due to the reaction of the body to the stent after it has been implanted.

As such there exists a need to encapsulate or otherwise shield or isolate such radiopaque coatings so as to maintain the stent's overall functionality and biocompatibility in spite of the use of any underlying radiopaque coatings.

Thus, there continues to be a need for an improved stent that has greater radiopacity yet maintains its overall functionality and biocompatibility. Such a stent would improve the visibility during insertion and deployment, increase the biocompatibility of its structural material, and help reduce the body's inflammatory response to the stent. The improved stent would also provide a platform for the application and adhesion of coatings that can deliver pharmacology locally and effectively to the vascular tissue bed with controlled, time-release qualities.

### SUMMARY OF THE INVENTION

One aspect of the invention provides a system for treating a vascular condition, including a catheter, a stent coupled to the catheter having a stent framework, a radiopaque oxide coating substantially covering at least an outer perimeter portion of the stent framework, and an encapsulant coating disposed on the radiopaque oxide coating.

Another aspect of the invention provides a drug-coated stent. The drug-coated stent includes a stent framework, a radiopaque oxide coating disposed on the stent framework, an encapsulant coating disposed on the radiopaque oxide coating, and a drug-polymer coating disposed on the encapsulant coating.

Another aspect of the invention provides a method of manufacturing a drug-coated stent. A radiopaque oxide coating is deposited onto an outer perimeter portion of a stent framework and an encapsulant coating is applied onto the radiopaque oxide coating.

The present invention is illustrated by the accompanying drawings of various embodiments and the detailed description given below. The drawings should not be taken to limit the invention to the specific embodiments, but are for explanation and understanding. The detailed description and drawings are merely illustrative of the invention rather than limiting, the scope of the invention being defined by the appended claims and equivalents thereof. The foregoing aspects and other attendant advantages of the present invention will become more readily appreciated by the detailed description taken in conjunction with the accompanying drawings.

### BRIEF DESCRIPTION OF THE DRAWINGS

Various embodiments of the present invention are illustrated by the accompanying figures, wherein:

FIG. 1 is an illustration of a system for treating a vascular condition including a catheter, a stent, a radiopaque oxide coating, an encapsulant coating, and a drug-polymer coating, in accordance with one embodiment of the current invention;

FIG. 2 is a cross-sectional view of a drug-coated stent with a radiopaque oxide coating, an encapsulant coating, and a drug-polymer coating, in accordance with one embodiment of the current invention;

FIG. 3 is a cross-sectional view of a drug-coated stent with a radiopaque oxide coating on an outer perimeter portion of a stent framework, an encapsulant coating, and a drug-polymer coating, in accordance with one embodiment of the current invention; and

FIG. 4 is a flow diagram of one embodiment of a method for manufacturing a drug-coated stent, in accordance with one embodiment of the current invention.

### DETAILED DESCRIPTION OF THE PRESENTLY PREFERRED EMBODIMENTS

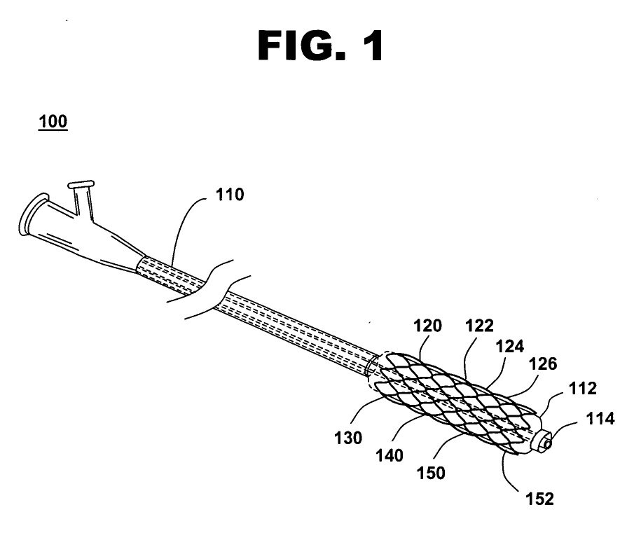

FIG. 1 is an illustration of a system for treating a vascular condition, including a catheter, a stent, a radiopaque oxide coating, an encapsulant coating, and a drug-polymer coating, in accordance with one embodiment of the present invention at 100. Vascular condition treatment system 100 includes a catheter 110, a stent 120 with a stent framework 122 coupled to catheter 110, a radiopaque oxide coating 130 substantially covering at least an outer perimeter portion 124 of stent framework 122, and an encapsulant coating 140 disposed on radiopaque oxide coating 130. Radiopaque coatings increase the visibility of stent framework 122 during deployment and post-insertion with conventional fluoroscopic and x-ray imaging techniques, particularly with stent designs having thinner struts and delicate latticework. Radiopaque coatings along the surfaces of stent framework 122, unlike radiopaque marker bands placed proximal and distal to stent 120, allow the clinician or physician to readily see the diameter and position of expandable stent 120 during its deployment within the vessel. The encapsulant and radiopaque coatings seal the surfaces of stent framework 122 and isolate the surfaces from body tissue. Encapsulant coating 140 may also provide an enhanced substrate for application and attachment of subsequent therapy layers.

One aspect of the invention is a system for treating coronary heart disease and other vascular conditions that use coated stents, which are deployed endovascularly by catheters. The stent coatings include a polymeric coating having one or more drugs with desired timed-release properties, a radiopaque oxide coating, and an encapsulant coating that serves as a primer or adhesion layer between the stent and the drug polymer.

Insertion of drug-coated stent 120 into a vessel in the body helps treat, for example, heart disease, various cardiovascular ailments, and other vascular conditions. Catheter-deployed stent 120 typically is used to treat one or more blockages, occlusions, stenoses, or diseased regions in the coronary artery, femoral artery, peripheral arteries, and other arteries in the body. Treatment of vascular conditions involves the prevention or correction of various ailments and deficiencies associated with the cardiovascular system, the cerebrovascular system, urinogenital systems, biliary conduits, abdominal passageways and other biological vessels within the body.

Catheter 110 of an exemplary embodiment of the present invention includes a balloon 112 that expands and deploys stent 120 within a vessel of the body. Stent 120 is coupled to catheter 110, and may be deployed by pressurizing a balloon coupled to the stent and expanding stent 120 to a prescribed diameter. A flexible guidewire traversing through a guidewire lumen 114 inside catheter 110 helps guide stent 120 to a treatment site, and once stent 120 is positioned, balloon 112 is inflated by pressurizing a fluid such as a contrast fluid that flows through a tube inside catheter 110 and into balloon 112. Stent 120 is expanded by balloon 112 until a desired diameter is reached, and then the contrast fluid is depressurized or pumped out, separating balloon 112 from deployed stent 120. Alternatively, catheter 110 may include a sheath that retracts to deploy a self-expanding version of stent 120.

Stent framework 122 includes a polymeric base or a metallic base such as stainless steel, nitinol, tantalum, MP35N alloy, platinum, titanium, a suitable biocompatible alloy, a suitable biocompatible material, and combinations thereof.

Radiopaque oxide coating 130 comprises a metal oxide such as iridium oxide. The metal oxide layer imparts a level of radiopacity to stent framework 122 that is higher than a bare metal framework, while rendering the outer surface more biocompatible than the outside surface of an unencapsulated stent. Radiopaque oxide coating 130 substantially covers the exterior surface or outer perimeter portion 124 of stent framework 122 and may also cover the interior surface or interior portion 126 of stent framework 122. In some cases, the struts and spars that form stent framework 122 are uniformly coated around the outside of each strut and spar with radiopaque oxide coating 130. In other cases, the outer perimeter or exterior surface is substantially covered with radiopaque oxide coating 130 and the interior surface towards the longitudinal central axis of stent 120 is void or minimally coated with radiopaque oxide coating 130. Radiopaque oxide coating 130 has a thickness, for example, between 0.2 micrometers (microns) and 1.5 microns or more to provide the desired radiopacity while adding minimal additional material to the stent framework. The radiopaque material of radiopaque oxide coating 130 may be encapsulated with encapsulant coating 140, providing improved biocompatibility, and forming a base or adhesion layer for additional drug-polymer layers.

Encapsulant coating 140 comprises, for example, parylene C or parylene N, which forms a protective conformal coating on the spars and struts of stent framework 122. Using conventional coating processes, a powdered form of parylene dimer is heated and vaporized, and then cracked in a vacuum at an elevated temperature to break the dimer into monomers. While stent 120 is in a coating chamber, the monomers deposit on the spars and struts of stent framework 122 and form into short segments of parylene C or are polymerized to form long polymeric chains of parylene N. The result is a relatively inert, uniform encapsulant coating 140 on top of radiopaque oxide coating 130 and on any exposed portions of stent framework 122. Encapsulant coating 140 can be used as an effective primer coating to promote adhesion between a metal stent surface and a subsequent polymer coating. The primer coating acts as a bridge between substrates and organic polymer coatings, with good adhesion properties to the metal and to a drug-polymer coating 150.

After encapsulant coating 140 is applied to stent 120 and dried, drug-polymer coating 150 may be disposed on encapsulant coating 140 to provide desired therapeutic properties. An exemplary drug-polymer coating 150 comprises one or more therapeutic agents 152 that are eluted with controlled time delivery after the deployment of stent 120 within the body. Therapeutic agent 152 is capable of producing a beneficial effect against one or more conditions including coronary restenosis, cardiovascular restenosis, angiographic restenosis, arteriosclerosis, hyperplasia, and other diseases or conditions.

For example, therapeutic agent 152 may be selected to inhibit or prevent vascular restenosis, a condition corresponding to a narrowing or constriction of the diameter of the bodily lumen where stent 120 is placed. Drug-polymer coating 150 may comprise, for example, an antirestenotic drug such as rapamycin, a rapamycin analogue, or a rapamycin derivative to prevent or reduce the recurrence or narrowing and blockage of the bodily vessel. Drug-polymer coating 150 may comprise an anti-cancer agent such as camptothecin or other topoisomerase inhibitors, an antisense agent, an antineoplastic agent such as triethylene thiophosphoramide, an antiproliferative agent, an antithrombogenic agent, an anticoagulant, an antiplatelet agent, an antibiotic, an anti-inflammatory agent, a steroid, a gene therapy agent, a therapeutic substance, an organic drug, a pharmaceutical compound, a recombinant DNA product, a recombinant RNA product, a collagen, a collagenic derivative, a protein, a protein analog, a saccharide, a saccharide derivative, a bioactive agent, a pharmaceutical drug, and combinations thereof. Therapeutic agent 152 may also include analogs and derivatives of these pharmaceutical compounds. Antioxidants may be beneficial for their antirestonotic properties and therapeutic effects.

The drugs can be encapsulated in drug-polymer coating 150 using a microbead, microparticle or nanoencapsulation technology with albumin, liposome, ferritin or other biodegradable proteins and phospholipids, prior to application on stent 120.

Drug-polymer coating 150 may soften, dissolve or erode from the stent such that at least one bioactive agent is eluted by surface erosion where the outside surface of the drug-polymer coating dissolves, degrades, or is absorbed by the body; or by bulk erosion where the bulk of the drug-polymer coating biodegrades to release the bioactive agent. Eroded portions of the drug-polymer coating 150 are absorbed by the body, metabolized, or otherwise expelled.

The elution rates of therapeutic agents 152 and total drug eluted into the body and the tissue bed surrounding the stent framework are based on the thickness of drug-polymer coating 150, the constituency of drug-polymer coating 150, the nature, distribution and concentration of therapeutic agents 152, the thickness and composition of any additional coatings, and other factors. An additional coating can be selected and disposed on drug-polymer coating 150 to provide a diffusion barrier for therapeutic agents 152 and to control the rate of drug elution.

Incorporation of a drug or other therapeutic agents 152 into drug-polymer coating 150 allows, for example, the rapid delivery of a pharmacologically active drug or bioactive agent within twenty-four hours following the deployment of a stent, with a slower, steady delivery of a second bioactive agent over the next three to six months. The therapeutic agent constituency in drug-polymer coating 150 may be, for example, between 0.1 percent and 50 percent or more of the drug-polymer coating by weight. Unlike drug-polymer coating 150 that are frequently eluted, metabolized, or discarded by the body, underlying encapsulant coating 140 and radiopaque oxide coating 130 often remain on stent framework 122.

One embodiment of drug-polymer coating 150 includes a polymeric matrix such as a caprolactone-based polymer or copolymer, and a cyclic polymer. The polymeric matrix may include various synthetic and non-synthetic or naturally occurring macromolecules and their derivatives. The polymeric matrix may include biodegradable polymers such as polylactide (PLA), polyglycolic acd (PGA) polymer, poly (e-caprolactone) (PCL), polyacrylates, polymethacryates, or other copolymers. The pharmaceutical drug may be dispersed throughout the polymeric matrix. The pharmaceutical drug or the bioactive agent may diffuse out from the polymeric matrix to elute the bioactive agent and into the biomaterial surrounding the stent.

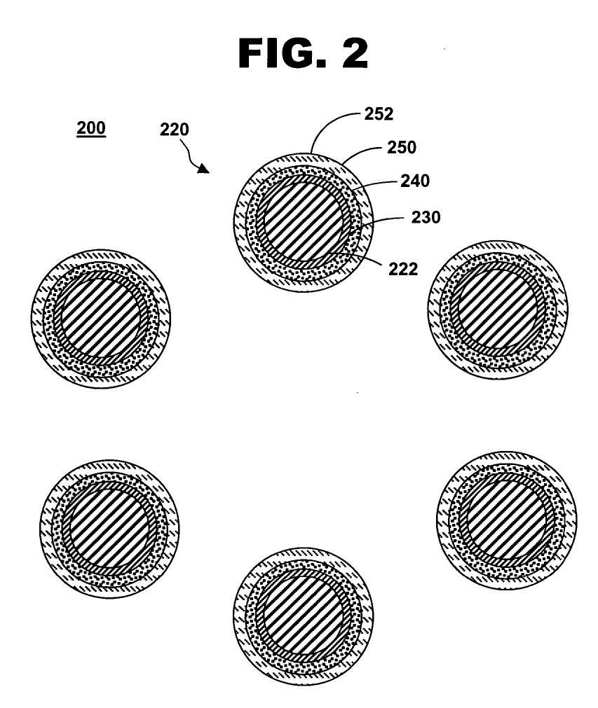

FIG. 2 is a cross-sectional view of a drug-coated stent with a radiopaque oxide coating, an encapsulant coating, and a drug-polymer coating, in accordance with one embodiment of the present invention at 200. Drug-coated stent 220 includes a stent framework 222. The stent coatings include a radiopaque oxide coating 230 disposed on stent framework 222, an encapsulant coating 240 disposed on radiopaque oxide coating 230, and an optional drug-polymer coating 250 disposed on encapsulant coating 240.

Stent framework 222 of stent 220 comprises a polymeric base or a metallic base such as stainless steel, nitinol, tantalum, MP35N alloy, platinum, titanium, a suitable biocompatible alloy, a suitable biocompatible material, and combinations thereof. To increase radiopacity, stent framework 222 is coated with a radiopaque metal oxide such as iridium oxide. The thickness of radiopaque oxide coating 230 ranges, for example, between 0.2 and 1.5 microns or more to achieve the desired radiopacity.

An encapsulant coating 240 including, for example, parylene C or parylene N covers radiopaque oxide coating 230 and any exposed portions of stent framework 222. A drug-polymer may be coated onto encapsulant coating 240.

Drug-polymer coating 250 includes a therapeutic agent 252 such as rapamycin, a rapamycin derivative, a rapamycin analogue, an antirestenotic drug, an anti-cancer agent, an antisense agent, an antineoplastic agent, an antiproliferative agent, an antithrombogenic agent, an anticoagulant, an antiplatelet agent, an antibiotic, an anti-inflammatory agent, a steroid, a gene therapy agent, a therapeutic substance, an organic drug, a pharmaceutical compound, a recombinant DNA product, a recombinant RNA product, a collagen, a collagenic derivative, a protein, a protein analog, a saccharide, a saccharide derivative, a bioactive agent, a pharmaceutical drug, and combinations thereof.

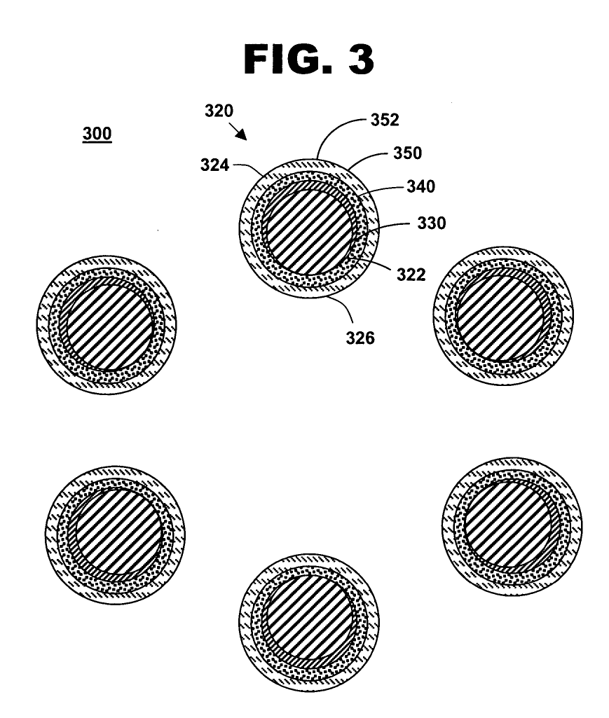

FIG. 3 is a cross-sectional view of a drug-coated stent with a radiopaque oxide coating on an outer perimeter portion of a stent framework, an encapsulant coating, and a drug-polymer coating, in accordance with one embodiment of the present invention at 300. A drug-coated stent 320 includes a stent framework 322. A radiopaque oxide coating 330 is disposed on stent framework 322 and an encapsulant coating 340 is disposed on radiopaque oxide coating 330. A drug-polymer coating 350 with one or more pharmaceutical agents 352 may be disposed on encapsulant coating 340.

Radiopaque oxide coating 330 substantially covers an outer perimeter portion 324 of stent framework 322. An interior portion 326 of stent framework 322 may be covered or uncovered with radiopaque oxide coating 330 depending on the application process. For example, a film of iridium oxide may be deposited on stent framework 322 as stent 320 is rotated about a mandrel in a vacuum deposition system, resulting in a larger thickness on outer perimeter portion 324 relative to interior portion 326. In other cases where the iridium oxide is electroplated, the thickness of radiopaque oxide coating 330 will be more uniform between outer perimeter portion 324 and interior portion 326. With vapor deposition techniques, subsequent coatings of the encapsulant material are substantially uniform in thickness about the struts and spars of stent framework 322. Drug-polymer coatings 350, which may coat stent framework 322 either uniformly or non-uniformly are applied on top of encapsulant coating 340 by such methods as dipping, spraying, painting or brushing.

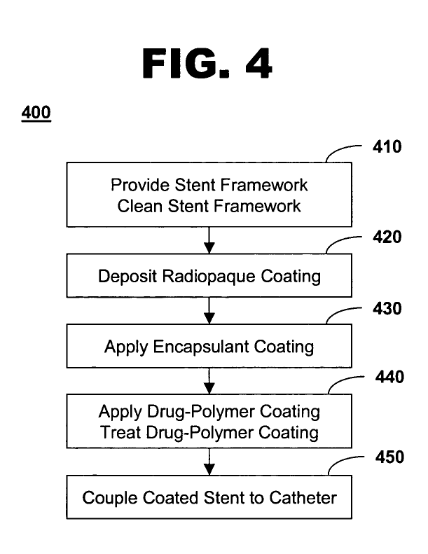

FIG. 4 is a flow diagram of one embodiment of a method for manufacturing a drug-coated stent with a radiopaque oxide layer and an encapsulant coating, in accordance with one embodiment of the present invention at 400.

A stent framework is provided and cleaned, as seen at block 410. Prior to the application of the radiopaque coating, the stent may be cleaned using, for example, degreasers, solvents, surfactants, de-ionized water or other cleaners, as is known in the art.

A radiopaque oxide coating is deposited onto an outer perimeter portion of a stent framework, as seen at block 420. The deposited radiopaque oxide comprises a radiopaque metal oxide coating such as iridium oxide, which is deposited using, for example, electroplating, sputter deposition, reactive sputtering, evaporation of iridium and subsequent oxidation of the iridium, and other plasma techniques. The thickness of the deposited radiopaque oxide coating is between, for example, 0.2 and 1.5 microns or more to provide sufficient radiopacity for viewing of the stent during deployment and inspection.

An encapsulant coating is applied onto the radiopaque oxide coating, as seen at block 430. The encapsulant coating may be applied to the stent framework using vapor deposition, dipping and drying, spraying, or other application techniques. An exemplary encapsulant coating comprises a biocompatible coating of parylene C or parylene N, which are applied using vapor deposition techniques whereby a parylene dimer is heated and evaporated. The heated parylene is injected into a vacuum environment at an elevated temperature where they form parylene monomers. The parylene monomers are transported to a coating chamber containing one or more stent frameworks, where the monomers deposit on the stent frameworks and form into short length chains of parylene C or polymerize into long-length chains of parylene N. The parylene C or parylene N is deposited until the desired thickness is reached. The stent frameworks are then removed from the coating chamber and cooled. A second coating step may be used to thicken the parylene coating when needed. The thickness of the encapsulant coating may range between 0.2 microns and 5.0 microns or greater in order to adequately coat the stent framework and to provide a satisfactory underlayer for subsequent drug-polymer application. The weight of the encapsulant coating depends on the diameter and length of the stent. Additional application steps may be included to reach the desired thickness of the primer coating.

After the encapsulant coating is applied, the stent may be packaged and shipped for use, or it may be coated further with a drug-polymer or another coating before being packaged and delivered. The optional drug-polymer coating is applied onto the encapsulant coating disposed on the stent framework and treated, as seen at block 440. The drug-polymer coating may be applied immediately after the encapsulant coating is applied. Alternatively, drug-polymer coatings may be applied to a stent with the encapsulant coating at a later time.

An exemplary drug polymer, which includes a polymeric matrix and one or more therapeutic compounds, is mixed with a suitable solvent to form a polymeric solution and is applied using an application technique such as dipping, spraying, paint, or brushing. During the coating operation, the drug-polymer adheres well to the encapsulant coating and any excess drug-polymer solution may be removed, for example, by being blown off. In order to eliminate or remove any volatile components, the polymeric solution is dried at room temperature or at elevated temperatures under dry nitrogen or other suitable environment. A second dipping and drying step may be used to increase the thickness of the drug-polymer coating, the thickness ranging between 1.0 microns and 200 microns or greater in order to provide sufficient and satisfactory pharmacological benefit.

The drug-polymer coating may be treated, for example, by heating the drug-polymer coating to a predetermined temperature to drive off any remaining solvent or to effect any additional crosslinking or polymerization. The drug-polymer coating may be treated with air drying or low-temperature heating in air, nitrogen, or other controlled environment.

The coated stent having the drug-polymer, encapsulant and radiopaque oxide coatings is coupled to a catheter, as seen at block 450. The coated stent may be integrated into a system for treating vascular conditions such as heart disease, by assembling the coated stent onto the catheter. Finished coated stents may be reduced in diameter, placed into the distal end of the catheter, and formed, for example, with an interference fit that secures the stent onto the catheter. The catheter along with the drug-coated stent may be sterilized and placed in a catheter package prior to shipping and storing. Additional sterilization using conventional medical means occurs before clinical use.

Although the present invention applies to cardiovascular and endovascular stents with timed-release pharmaceutical drugs, the use of radiopaque oxides and encapsulant coatings under polymer-drug coatings may be applied to other implantable and blood-contacting biomedical devices such as coated pacemaker leads, microdelivery pumps, feeding and delivery catheters, heart valves, artificial livers, and other artificial organs.

1. A system for treating a vascular condition having a stent mounted to a catheter, the stent having a radiopaque oxide coating added to its surface so as to enhance the radiopacity of the stent, comprising:
a catheter;
a stent coupled to the catheter, the stent including a stent framework;
a radiopaque oxide coating substantially covering at least an outer perimeter portion of the stent framework; and
an encapsulant coating disposed on the radiopaque oxide coating so as to render the radiopaque oxide coating less reactive or fragile.

2. The system of claim 1 wherein the catheter includes a balloon used to expand the stent.

3. The system of claim 1 wherein the catheter includes a sheath that retracts to allow expansion of the stent.

4. The system of claim 1 wherein the stent framework comprises a metallic base.

5. The system of claim 4 wherein the metallic base is selected from the group consisting of stainless steel, nitinol, tantalum, MP35N alloy, platinum, titanium, a suitable biocompatible alloy, a suitable biocompatible material, and a combination thereof.

6. The system of claim 1 wherein the stent framework comprises a polymeric base.

7. The system of claim 1 wherein the radiopaque oxide coating comprises iridium oxide.

8. The system of claim 1 wherein the radiopaque oxide coating has a thickness between 0.2 and 1.5 microns.

9. The system of claim 1 wherein the encapsulant coating comprises one of parylene C and parylene N.

10. The system of claim 1 further comprising:
a drug-polymer coating disposed on the encapsulant coating, the drug-polymer coating including a therapeutic agent.

11. The system of claim 10 wherein the therapeutic agent is selected from the group consisting of rapamycin, a rapamycin analogue, a rapamycin derivative, an antirestenotic drug, an anti-cancer agent, an antisense agent, an antineoplastic agent, an antiproliferative agent, an antithrombogenic agent, an anticoagulant, an antiplatelet agent, an antibiotic, an anti-inflammatory agent, a steroid, a gene therapy agent, a therapeutic substance, an organic drug, a pharmaceutical compound, a recombinant DNA product, a recombinant RNA product, a collagen, a collagenic derivative, a protein, a protein analog, a saccharide, a saccharide derivative, a bioactive agent, a pharmaceutical drug, and a combination thereof.

12. A drug-coated stent, comprising:
a stent framework;
a radiopaque oxide coating disposed on the stent framework;
an encapsulant coating disposed on the radiopaque oxide coating; and
a drug-polymer coating disposed on the encapsulant coating.

13. The drug-coated stent of claim 12 wherein the stent framework comprises a metallic base.

14. The drug-coated stent of claim 13 wherein the metallic base is selected from the group consisting of stainless steel, nitinol, tantalum, MP35N alloy, platinum, titanium, a suitable biocompatible alloy, a suitable biocompatible material, and a combination thereof.

15. The drug-coated stent of claim 12 wherein the stent framework comprises a polymeric base.

16. The drug-coated stent of claim 12 wherein the radiopaque oxide coating comprises iridium oxide.

17. The drug-coated stent of claim 12 wherein the radiopaque oxide coating has a thickness between 0.2 and 1.5 microns.

18. The drug-coated stent of claim 12 wherein the encapsulant coating comprises one of parylene C and parylene N.

19. The drug-coated stent of claim 12 wherein the drug-polymer coating comprises a therapeutic agent.

20. The drug-coated stent of claim 19 wherein the therapeutic agent is selected from the group consisting of rapamycin, a rapamycin analogue, a rapamycin derivative, an antirestenotic drug, an anti-cancer agent, an antisense agent, an antineoplastic agent, an antiproliferative agent, an antithrombogenic agent, an anticoagulant, an antiplatelet agent, an antibiotic, an anti-inflammatory agent, a steroid, a gene therapy agent, a therapeutic substance, an organic drug, a pharmaceutical compound, a recombinant DNA product, a recombinant RNA product, a collagen, a collagenic derivative, a protein, a protein analog, a saccharide, a saccharide derivative, a bioactive agent, a pharmaceutical drug, and a combination thereof.

21. A method of manufacturing a drug-coated stent, comprising:
depositing a radiopaque oxide coating onto an outer perimeter portion of a stent framework;
applying an encapsulant coating onto the radiopaque oxide coating.

22. The method of claim 21 wherein the deposited radiopaque oxide coating comprises iridium oxide.

23. The method of claim 21 wherein the deposited radiopaque oxide coating has a thickness between 0.2 and 1.5 microns.

24. The method of claim 21 wherein the applied encapsulant coating comprises one of parylene C and parylene N.

25. The method of claim 21 further comprising;
applying a drug-polymer coating onto the encapsulant coating disposed on the stent framework; and
treating the drug-polymer coating.

26. The method of claim 25 wherein the drug-polymer coating is applied using an application technique selected from the group consisting of dipping, spraying, painting, and brushing.

27. The method of claim 25 wherein the drug-polymer coating is treated by heating the drug-polymer coating to a predetermined temperature.
